SEQUENCE LISTING (18 sequences)

SEQ ID NO: 1 - DNA, 66 bp, Artificial Sequence
  Description of Artificial Sequence Synthetic oligonucleotide
accgagagagattcgcactcaatattcaagagatattgagtgcgaatctctctctttttatcgatg

SEQ ID NO: 2 - DNA, 66 bp, Artificial Sequence
  Description of Artificial Sequence Synthetic oligonucleotide
accgagatcgccacctacaggaaattcaagagatttcctgtaggtggcgatctctttttatcgatg

SEQ ID NO: 3 - DNA, 21 bp, Artificial Sequence
  Description of Artificial Sequence Synthetic oligonucleotide
gagagagattcgcactcaata

SEQ ID NO: 4 - DNA, 21 bp, Artificial Sequence
  Description of Artificial Sequence Synthetic oligonucleotide
tattgagtgcgaatctctctc

SEQ ID NO: 5 - DNA, 21 bp, Artificial Sequence
  Description of Artificial Sequence Synthetic oligonucleotide
gagatcgccacctacaggaaa

SEQ ID NO: 6 - DNA, 21 bp, Artificial Sequence
  Description of Artificial Sequence Synthetic oligonucleotide
tttcctgtaggtggcgatctc

SEQ ID NO: 7 - DNA, 68 bp, Artificial Sequence
  Description of Artificial Sequence Synthetic oligonucleotide
accgaatggtacaagtccaggtttgttcaagagacaaacttggacttgtaccattctttttctcgagg

SEQ ID NO: 8 - DNA, 66 bp, Artificial Sequence
  Description of Artificial Sequence Synthetic oligonucleotide
accgagagaaattgcaggaggagattcaagagatctcctcctgcaatttctctctttttctcgagg

SEQ ID NO: 9 - DNA, 22 bp, Artificial Sequence
  Description of Artificial Sequence Synthetic oligonucleotide
gaatggtacaagtccaggtttg

SEQ ID NO: 10 - DNA, 22 bp, Artificial Sequence
  Description of Artificial Sequence Synthetic oligonucleotide
caaacttggacttgtaccattc

SEQ ID NO: 11 - DNA, 21 bp, Artificial Sequence
  Description of Artificial Sequence Synthetic oligonucleotide
gagagaaattgcaggaggaga

SEQ ID NO: 12 - DNA, 21 bp, Artificial Sequence
  Description of Artificial Sequence Synthetic oligonucleotide
tctcctcctgcaatttctctc

SEQ ID NO: 13 - DNA, 21 bp, Artificial Sequence
  Description of Combined DNA/RNA Molecule Synthetic oligonucleotide
gaaugguacaaauccaagutt

SEQ ID NO: 14 - DNA, 20 bp, Artificial Sequence
  Description of Combined DNA/RNA Molecule Synthetic oligonucleotide
acuuggauuuguaccauutt

SEQ ID NO: 15 - DNA, 21 bp, Artificial Sequence
  Description of Combined DNA/RNA Molecule Synthetic oligonucleotide
auggaagagaacuuugccgtt

SEQ ID NO: 16 - DNA, 21 bp, Artificial Sequence
  Description of Combined DNA/RNA Molecule Synthetic oligonucleotide
cggcaaaguucucuuccautt

SEQ ID NO: 17 - DNA, 21 bp, Artificial Sequence
  Description of Combined DNA/RNA Molecule Synthetic oligonucleotide
uaccaagaccugcucaaugtt

SEQ ID NO: 18 - DNA, 21 bp, Artificial Sequence
  Description of Combined DNA/RNA Molecule Synthetic oligonucleotide
cauugagcaggucuugguatt

(19) United States
(12) Patent Application Publication
(10) Pub. No.: US 2010/0098664 A1
(43) Pub. Date: Apr. 22, 2010

(54) Lentiviral vectors allowing RNAi mediated inhibition of GFAP and vimentin expression

(76) Inventors: Mathieu Jean-Francois Desclaux, Paris (FR); Jacques Mallet, Paris (FR); Alain Privat, St Clement de Riviere (FR); Minerva Gimenez y Ribotta, Alicante (ES)

Correspondence Address: NIXON & VANDERHYE, PC, 901 NORTH GLEBE ROAD, 11TH FLOOR, ARLINGTON, VA, 22203, US

(21) Appl. No.: 11/987,267

(22) Filed: Nov. 28, 2007

(51) Int. Cl. A61K 35/76; C12N 7/01

(52) U.S. Cl. 424/93.2; 435/235.1

(57) ABSTRACT

The present invention relates to method for preventing, treating or alleviating a central nervous system (CNS) disorder using a non replicative lentivirus comprising a lentiviral genome comprising a nucleic acid sequence producing at least one functional miRNA, at least one functional shRNA and/or at least one functional siRNA, preferably derived from said shRNA, said miRNA, shRNA or siRNA being designed to silence the expression of a gene that encodes a protein of the astrocyte cytoskeleton. The present invention further relates to compositions and kits comprising such a lentivirus as well as to uses thereof.

### FIELD OF THE INVENTION

The present invention relates to methods of inhibiting the expression of a gene encoding a protein of the astrocyte cytoskeleton, said protein being at least partly responsible for the formation of a glial scar occurring in certain conditions where the Central Nervous System (CNS) is damaged. Generally, the present methods involve the use of a lentiviral vector comprising a lentiviral genome comprising a nucleic acid sequence producing at least one functional micro RNA (miRNA), at least one functional short-hairpin RNA (shRNA) and/or at least one functional siRNA, preferably derived from said shRNA, said miRNA, shRNA and siRNA being designed to silence the expression of a gene that encodes a protein of the astrocyte cytoskeleton, in order to favour axonal regeneration.

### BACKGROUND

The axonal regeneration of injured and/or deteriorated neurons of the central nervous system constitutes a major stake in the elaboration of therapies.

The limited capacity of the adult CNS neurons to regenerate is, in particular, associated with the installation of a non permissive cellular environment, hostile to such a regeneration (Yiu et al., 2006, Fawcett et al., 2006, Fawcett and Asher 1999). Two types of events are responsible for the installation of this hostile environment: production of myelin-associated inhibitory factors, such as Nogo, MAG (myelin-associated glycoprotein) and OMgp (oligodendrocyte myelin glycoprotein) proteins, resulting from the degradation of the myelin sheath and of the accompanying oligodendrocytes, and formation of a glial scar essentially made of reactive astrocytes secreting inhibitory proteoglycans.

The glial scar, in particular, constitutes a major physical obstacle for axonal regeneration in conditions where the Central Nervous System (CNS) is damaged (Fawcett et al., 1999). The glial scar is mainly the consequence of astrocytes reactivity, a phenomenon resulting in astrocytes hyperplasia and hypertrophy.

Astrocytes reactivity is characterized, on the biochemical level, by the surexpression of at least two proteins of the astrocyte cytoskeleton, the glial fibrillary acidic protein (GFAP) and Vimentin, which are biochemical hallmarks of the hypertrophy of the reactive astrocytes.

Matrix proteoglycans, and more specifically chondroitin sulphate proteoglycans (CSPG), are other essential elements of the glial scar, which are synthesized by different types of glial cells during reactive gliosis (Silver and Miller, 2004). Recently, a strategy was developed to improve axonal regeneration through disintegration of CSPG thanks to a specific enzyme, chondroitinase ABC, which was injected directly into the scar tissue. This enzyme separates glycosaminoglycans from the protein core of CSPG and thus eliminates their negative influence on axonal regrowth (Bradbury et al, 2002, Moon et al, 2001). However, chondroitinase cannot be used as a therapeutic tool due to its high intrinsic toxicity for other cell components and due to its poor stability in time and space.

Other proteins of the intracellular matrix or of the cell surface are also involved into cellular interactions, and namely axon/glia relationships. One independent approach was that of Rutishauser, who over expressed the sialic acid component (PSA) which, when associated to the N-Cam protein, improves the plasticity of regenerating axons (El Maalouf et al, 2006). This condition mimics that of the foetal environment, where most of N-Cam is polysialylated. No improvement of function in animal models has however been reported to date using viral vectors overexpressing PSA.

Several myelin-associated molecules have been identified, as explained previously, as inhibitors of axonal regrowth.

The most studied is Nogo, which has been identified as such thanks to an antibody named IN-1, which was found to neutralize the inhibition provided in vitro by myelin on axonal elongation (Caroni and Schwab, 1988). Later-on this antibody was found to induce some regeneration of the corticospinal tract after a surgical section in adult rats (Schnell and Schwab, 1990, Brosamle et al, 2000). In this model, some recovery of motor functions was later described (Bregman et al, 1995). Since then, several groups attempted to generate transgenic animals with the deletion of the genes coding for the Nogo receptor or for the protein. The conclusions regarding axonal regeneration were contradictory from one author to another (Kim et al, 2003, Simoen et al, 2003, Zheng et al, 2003). Schwab has since extended his study using a NogoA antibody (Leibscher et al, 2005, Freund et al, 2006, 2007), and a phase 1 clinical trial has been launched recently using said antibody. Nogo antibodies are however associated with strong risks of immune reactions.

The other identified Myelin-associated inhibitors, MAG (myelin-associated glycoprotein) and OMpg (oligodendrocyte myelin glycoprotein), apparently share a common receptor with Nogo, semaphorin 4D and ephrin B3 (Yiu et al, 2006, Fawcett et al, 2006).

A mouse knocked out for the gene encoding MAG failed to show any axonal regeneration. (Bartsch et al, 1995).

Works performed until now focused on the identification of the different factors responsible for the glial environment induced inhibition of axonal regeneration, and on the understanding of each of said factors respective effects in this mechanism. Therapeutic strategies implying Chondroitinase ABC, anti Nogo antibodies, PSA or antiproliferative agent (purine analog), such as Ribavirin (Pekovic et al, 2005), have been suggested but each found associated to undesirable effects.

In order to more specifically inhibit the elements responsible for the hypertrophic part of astrocytes reactivity, transgenic mice were generated in which the genes coding for GFAP and vimentin were knocked-out. These knocked-out (KO) mice were first used to develop in vitro models of co-culture of wild type foetal neurons with transgenic reactive astrocytes, in order to appreciate, in a simplified system, the influence of the absence of these two proteins on neuron survival and neurite extension. The absence of GFAP alone, or of both proteins, allowed an increased neuronal survival and a profuse neurite extension. In addition, the expression of several proteoglycans on the surface of transgenic astrocytes was found to mimic that of immature astrocytes (the so called radial glia) which serve as a substrate and guide immature neurons during embryogenesis. Thus, GFAP appeared as a key protein in the control of astrocytes reactivity (Menet et al, 2001). These animals were then used to analyze the possible influence of the absence of these proteins on actual axonal regeneration in a model of spinal cord lesion. Control and mutant mice underwent a lateral hemisection of the spinal cord which induced a total paralysis of the hind limb on the lesion side. When compared with controls, double KO mice presented a reduction of the glial scar (which was apparent as soon as three days after the lesion), and a substantial regeneration, on their specific targets, of two systems of axons projecting respectively from the cerebral cortex and the brain stem (which was apparent after two weeks). Interestingly, when challenged with a motor task (grid walk), transgenic mice improved significantly their scores, after two weeks, whereas controls tended to deteriorate further (Menet et al, 2003).

There is an obvious need for a safe and efficient therapeutic strategy, and in particular for new tools that are able to achieve effective and specific inhibition of undesirable expressions to thereby promote axonal regeneration, in the context of a CNS disorder, without being compromised by serious unwanted side effects. Such tools and prophylactic or therapeutic methods are the subject of the invention.

### SUMMARY OF THE INVENTION

Inventors have now discovered that it is possible to safely, efficiently and stably silence genes that encode factors inducing formation of a glial scar, and thereby promote axonal regeneration in the context of a CNS disorder.

The present invention relates to compounds, compositions, and methods useful for modulating the expression and activity of protein of the astrocyte cytoskeleton by RNA interference (RNAi) using small nucleic acid molecules, such as short interfering RNA (siRNA), as specified in the attached claims, incorporated herein by reference.

It is indeed herein demonstrated that RNA interference (RNAi) constitutes a powerful tool to efficiently and specifically silence, on the post-transcriptional level, the expression of a protein of the astrocyte cytoskeleton. Herein described are lentiviral vectors designed to reach this aim in a safe and controlled manner.

The present disclosure in particular provides a non replicative lentivirus comprising a lentiviral genome comprising a nucleic acid sequence producing at least one functional miRNA, at least one functional short-hairpin RNA (shRNA) and/or at least one functional siRNA, preferably derived from said shRNA, said miRNA, shRNA and siRNA being designed to silence the expression of a gene that encodes a protein of the astrocyte cytoskeleton, said lentivirus being pseudotyped for the selective transfer of the lentiviral genome into cells of the central nervous system, in particular in glial cells, preferably in astrocytes.

The disclosure also provides compositions, in particular pharmaceutical compositions comprising one or more of the present lentiviruses and a pharmaceutically acceptable carrier or excipient.

Also provided are methods of preventing, treating or alleviating a central nervous system (CNS) disorder in an animal subject, preferably a human, wherein glial scar is believed to play a role in the pathogenesis of the disorder, said methods comprising administering to said subject a pharmaceutical composition comprising a defective lentivirus as mentioned previously, and a pharmaceutically acceptable carrier or excipient.

In another aspect, the present disclosure provides kits comprising any one or more of the herein-described lentivirus or compositions. Typically, the kit also comprises instructions for using the lentivirus or compositions according to the present methods.

### BRIEF DESCRIPTION OF THE DRAWINGS

FIG. 1: RNA interference pathways (from Dykxhoorn et al. 2003)
a) Structure of a short interfering RNA (siRNA). Molecular of an siRNA include 5′ phosphorylated ends, a 19 to 22 nucleotides duplexed region and 2 nucleotides unpaired and unphosphorylated 3′ ends that are characteristic of RNAse III cleavage products.
b) siRNA pathway. In an initiation phase, double stranded RNA (dsRNA) is cleaved by the enzyme DICER, giving rise to siRNA duplex. In an activation phase, the siRNA duplex is associated to the enzymatic complex that leads to recognition of the mRNA target and enzymatic degradation of the mRNA target.
c) miRNA pathway. DICER also cleaves the ˜70 nucleotides hairpin miRNA precursor to produce ˜22 nucleotides miRNA.

FIG. 2: Methods to generate siRNAs in vivo from plasmidic or viral vectors
A) sense and antisense strands of the siRNA are expressed from tandem polIII promoters
B) short hairpin RNA (shRNA) is expressed from a singe polIII promoter
C) Imperfect duplex hairpin, based on pre-miRNA is expressed from polII promoter and is processed by DICER into a mature miRNA

FIG. 3: Screening of shRNAs targeting GFAP and Vimentin—Analysis of GFAP-GFP and Vimentin-GFP expression by Fluorescent Activated Cell Sorting (FACS), 72 h after cotransfection
A) Percentages of GFP positives cells were measured for the different shGFAP contructs
B) Mean fluorescence intensity was measured for the different shGFAP contructs
C) Percentages of GFP positives cells were measured for the different shVIM constructs
D) Mean fluorescence intensity was measured for the different shVIM contructs

FIG. 4: Design of a lentiviral vector allowing expression of shGFAP or shVIM. LTR, ψ, and Flap are HIV-1 derived sequences (the LTRs, the packaging sequence, and the central Flap element, respectively). P_{U6} and P_{PGK} are respectively the human U6 promoter and the ubiquitous PGK promoter. GFP is the coding sequence of Green Fluorescent Protein, and WPRE, the Woodchuck hepatitis virus responsive element.

FIG. 5: Lentiviral mediated silencing of GFAP and Vimentin expression by RNAi in primary astrocytes. Astrocytes were transduced with various amounts of vectors, expressed in number of viral particles per seeded cell (MOI=multiplicity of infection).

FIG. 6: Effects of the lentiviral vectors on neuronal survival and neurite growth.
A) Neuronal survival was assayed by counting the βIII-tubulin positive cells.
B) Neurite outgrowth was evaluated by measuring the surface occupied by perikarya and neurites per neuron.
   NT=non transduced cells. Lv-PGK-GFP, Lv-shRANDOM and Lv-shG1 are used as control vectors (*p<0.05; **p<0.01; ***p<0.001; Mann-Whitney test).

FIG. 7: Schematic representation of the intraparenchymal injection of the Lv-shGFAP and Lv-shVIM lentiviral vectors
A) Representation of the injections sub-sites
B) Representation of the injections sites

FIG. 8: In vivo inhibition of GFAP expression by the lentiviral vector Lv-shGFAP. These pictures presente GFP and GFAP immunostaining on spinal cord frozen sections of a previously hemisected mouse. The transduced area is visualized by GFP immunostaining (green) and GFAP immunostaining (red).

A) and B): Two weeks after transduction, GFAP expression is decreased in spinal cord transduced with Lv-shGFAP (A) when compared with the control Lv-PGK-GFP vector (B).
C) and D): Five weeks after transduction, GFAP expression is decreased in spinal cord transduced with Lv-shGFAP (C) when compared with the control Lv-PGK-GFP vector (D).

FIG. 9: In vivo inhibition of Vimentin expression by the lentiviral vector Lv-shVIM. These pictures presente GFP and VIM immunostaining on spinal cord frozen sections of a previously hemisected mouse. The transduced area is visualized by GFP immunostaining (green) and GFAP immunostaining (red).

A) and B): five weeks after transduction, Vimentin expression is decreased in spinal cord transduced with Lv-shVIM (A) when compared with the control Lv-shRANDOM vector (B).

FIG. 10: Effects of the Lv-shGFAP and Lv-shVIM vectors on functional recovery in an animal model of spinal cord injury.

The different vectors were injected in mice in which inventors have performed a total unilateral hemisection of the spinal cord. Grid runway test was performed 5 weeks after lesion and injection of the lentiviral vectors. PBS, Lv-PGK-GFP and Lv-H1-shRANDOM are used as controls. Recuperation was measured by the difference between faults number at 2 weeks and faults number at five weeks. Statistical significancy was evaluated by non-parametric Mann-Whitney test (*p<0.05)

FIG. 11: Analysis of serotonergic regrowth in the ventral horn after hemisection and injection of the Lv-shGFAP and Lv-shVIM vectors. % of the surface occupied by serotonergic fibres in the ventral horn of the lesioned side was evaluated for each vectors condition in comparison with the non-lesioned side.

### DETAILED DESCRIPTION OF THE INVENTION

### Definitions

In describing, exemplifying and claiming the present invention, the following terminology will be used in accordance with the definitions set out below. Where not otherwise indicated, the terms are intended to have the meaning generally recognized in the art.

By “GFAP” is meant GFAP peptide or protein or a naturally occurring fragment thereof, wherein the peptide or protein is encoded by the GFAP gene.

By “vimentin” is meant vimentin peptide or protein or a naturally occurring fragment thereof, wherein the peptide or protein is encoded by the vimentin gene.

By “RNA” is meant a molecule comprising at least one ribonucleotide residue. By “ribonucleotide” is meant a nucleotide with a hydroxyl group at the 2′ position of a-D-ribo-furanose moiety. The terms include double-stranded RNA, single-stranded RNA, isolated RNA such as partially purified RNA, essentially pure RNA, synthetic RNA, recombinantly produced RNA, as well as comprising non-standard nucleotides, such as non-naturally occurring nucleotides or chemically synthesized nucleotides or deoxynucleotides.

The term “short interfering nucleic acid”, “siRNA”, “short interfering RNA”, “siRNA”, “short interfering nucleic acid molecule”, “short interfering oligonucleotide molecule”, “miRNA”, “micro RNA” as used herein refers to any nucleic acid molecule capable of mediating RNA interference “RNAi” or gene silencing in a sequence-specific manner. RNA interference (RNAi) describes a process wherein double-stranded RNA (dsRNA), when present inside a cell, inhibits expression of an endogenous gene that has an identical or nearly identical sequence to that of the dsRNA. Inhibition is caused by the specific degradation of the messenger RNA (mRNA) transcribed from the target gene. In greater detail, RNA interference describes a process of sequence-specific post-transcriptional gene silencing in animals mediated by the expression of “short interfering RNAs” (siRNAs) after in situ cleavage (Brummelkamp et al., 2002). The initial basic process involves double stranded RNA (dsRNA) that is/are processed by cleavage into shorter units (the so called siRNA) that guide recognition and targeted cleavage of homologous target messenger RNA (mRNA) (see FIG. 1a).

The method does not require the time-consuming genetic manipulations needed for classical gene knock-out strategies and has therefore emerged as a valuable tool in molecular genetics that may also be applied to human therapy.

The currently known mechanism of RNAi can be described as follows:

The processing of dsRNA into siRNAs, which in turn induces degradation of the intended target mRNA, is a two-step RNA degradation process. The first step involves a dsRNA endonuclease (ribonuclease III-like; RNase III-like) activity that processes dsRNA into smaller sense and antisense RNAs which are most often in the range of 21 to 25 nucleotides (nt) long, giving rise to the so called short interfering RNAs (siRNAs). This RNase III-type protein is termed “Dicer”. In a second step, the antisense siRNAs produced combine with, and serve as guides for, a different ribonuclease complex called RNA-induced silencing complex (RISC), which allows annealing of the siRNA and the homologous single-stranded target mRNA, and the cleavage of the target homologous single-stranded mRNAs. Cleavage of the target mRNA has been observed to place in the middle of the duplex region complementary to the antisense strand of the siRNA duplex and the intended target mRNA (see FIG. 1b) (Dykxhoorn et al., 2003 for review).

Micro RNAs (miRNAs) constitute non coding RNAs of 21 to 25 nucleotides, which controls genes expression at post-transcriptional level. miRNAs are synthesized from ARN polymerase II or ARN polymerase III in a pre-miRna of 125 nucleotides. Pre-miRNA are cleaved in the nucleus by the enzyme Drosha, giving rise to a precursor called imperfect duplex hairpin RNA (or miRNA-based hairpin RNA). These imperfect duplex hairpin RNAs are exported from the nucleus to the cytoplasm by exportin-5 protein, where it is cleaved by the enzyme DICER, giving rise to mature miRNAs. miRNAs combine with RISC complex which allows total or partial annealing with the homologous single-stranded target mRNA. Partial annealing with the mRNA leads to the repression of protein translation, whereas total annealing leads to cleavage of the single-stranded mRNA (see FIG. 1c). (Dykxhoorn et al., 2003 for review).

At least three methods to generate RNAi-mediated gene silencing in vivo are known and usable in the context of the present invention (Dykxhoorn et al., 2003 for review): siRNAs with a single sequence specificity can be expressed in vivo from plasmidic or viral vectors using:
Tandem polymerase III promoter that express individual sense and antisense strands of the siRNAs that associate in trans (see figure A3-A)
a single polymerase III promoter that expresses short hairpin RNAs (shRNAs) (see figure A3-B)
a single polymerase II promoter that expresses an imperfect duplex hairpin RNA (pre-miRNA) which is processed by DICER giving rise to a mature miRNA (see figure A3-C).

By “antisense strand” is meant a nucleotide sequence of a siRNA molecule having complementarity to a sense region of the siRNA molecule. In addition, the antisense strand of a siRNA molecule comprises a nucleic acid sequence having homology with a target nucleic acid sequence.

By “sense strand” is meant a nucleotide sequence of a siRNA molecule having complementarity to an antisense region of the siRNA molecule.

By “modulate” and “modulation” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit” and within the scope of the invention, the preferred form of modulation is inhibition but the use of the word “modulate” is not limited to this definition.

By “inhibit”, “silence” or “down regulate” it is meant that the levels of expression product or level of RNAs or equivalent RNAs encoding one or more gene products is reduced below that observed in the absence of the nucleic acid molecule of the invention. In one embodiment, inhibition with a nucleic acid molecule capable of mediating RNA interference (siRNA, shRNA, miRNA) preferably is below that level observed in the presence of an inactive or attenuated molecule that is unable to mediate an RNAi response.

By “target protein” is meant any protein whose expression or activity is to be modulated. Preferred target proteins are GFAP and vimentine.

By “target nucleic acid” or “target gene” is meant any nucleic acid sequence whose expression or activity is to be modulated. The target nucleic acid can be DNA or RNA. In the context of the invention, the “target gene” is a gene that encodes a protein of the astrocyte cytoskeleton, typically the GFAP or the vimentine gene.

By “subject” is meant an organism, which is a donor or recipient of explanted cells or the cells themselves. “Subject” also refers to an organism to which the nucleic acid molecules of the invention can be administered. The subject may be a non-human animal, preferably a mammal. The preferred subject is a human subject.

Glial fibrillary acidic protein (GFAP) is a 55 kDa cytosolic protein that is a major structural component of astroglial filaments and is the major intermediate filament protein in astrocytes. GFAP is specific to astrocytes. This protein helps to maintain astrocyte mechanical strength and shape. This protein is involved in reactive astrocyte hypertrophy.

Vimentin is a 57 kDa cytosolic protein that is a major structural component of astroglial filaments and a major intermediate filament protein in astrocytes. Vimentin is specifically re-expressed in reactive astrocytes after CNS injury. This protein is involved in reactive astrocyte hypertrophy.

### New Approach for Axonal Regeneration:

The brain contains two major types of cells: neurons and glial cells. Neurons are the most important cells in the brain, and are responsible for maintaining communication within the brain via electrical and chemical signalling. Glial cells function mainly as structural components of the brain, and they are approximately 10 times more abundant than neurons. Glial cells of the central nervous system (CNS) are astrocytes and oligodendrocytes.

Astroglial cells respond to trauma and ischemia with reactive gliosis (also called “astrocytic activation”), a reaction characterized by increased astrocytic proliferation and hypertrophy. Although beneficial to a certain extent, excessive gliosis may be detrimental, contributing to neuronal death in neurodegenerative diseases and in SNC trauma.

Astrocytic activation evidenced by increased glial fibrillary acidic protein has been found for example in multiple sclerosis (Malmestrom C, Haghighi S, Rosengren L, Andersen O, Lycke J: Neurofilament light protein and glial fibrillary acidic protein as biological markers in MS. Neurology 2003; 61:1720-1725), temporal lobe epilepsy (Briellmann R S, Kalnins R M, Berkovic S F, Jackson G D: Hippocampal pathology in refractory temporal lobe epilepsy: T2-weighted signal change reflects dentate gliosis. Neurology 2002; 58:265-271), amyotrophic lateral sclerosis (Lexianu M, Kozovska M, Appel S H: Immune reactivity in a mouse model of familial ALS correlates with disease progression. Neurology 2001; 57:1282-1289), systemic lupus erythematosus (Trysberg E, Nylen K, Rosengren L E, Tarkowski A: Neuronal and astrocytic damage in systemic lupus erythematosus patients with central nervous system involvement. Arthritis Rheum 2003; 48:2881-2887), human immunodeficiency virus dementia, Alzheimer's dementia, traumatic injury, Parkinson disease (Teismann and Schulz, 2004) and Alzheimer's disease (Sjobeck and Englund, 2003).

The present disclosure provides a novel strategy of axonal regeneration based on the neutralization, via gene transfer, of the elements which, during reactive astrocytic gliosis, are responsible for the formation of a biochemical and physical barrier, the so called “glial scar”, composed of reactive astrocytes and proteoglycans. The biochemical hallmark of astrogliosis is the massive upregulation of the intermediate filament proteins (IF) GFAP and vimentin.

Inventors herein demonstrate that said proteins of the astrocyte cytoskeleton are appropriate and valuable targets in the context of therapy, in particular of human therapy. Inventors herein provide vectors carrying nucleic acid molecule mediating RNAi, in particular nucleic acid molecules producing miRNA, shRNA and/or siRNA molecules that down regulate expression of proteins of the astrocyte cytoskeleton by RNA interference. The inventors herein demonstrate the beneficial impact of these vectors on health in several animal models of CNS disorder. The vectors were shown to be effective in vitro, as well as in vivo in animal models (see experimental part of the present application).

The vectors benefit from the technology of ribonucleic acid interference (RNAi), which is described above in great details.

Employing siRNAs in living animals, especially humans, was a challenge, since siRNAs show different effectiveness in different cell types in a manner yet poorly understood: some cells respond well to siRNAs and show a robust knockdown, others show no such knockdown (even despite efficient transfection). However it was a successful approach, which proved to be both safe and very efficient.

### Nucleic Acid Molecules Capable of Mediating RNA Interference

Preferred molecules capable of mediating RNA interference advantageously down regulate at least 60%, preferably at least 70%, preferably at least 80%, even more preferably at least 90%, of the target protein expression.

Preferred shRNA designed to silence a gene encoding GFAP are identified below (sequences in black design the siRNA sequence produced after cleavage of the shRNA by DICER):

Murine genome targeting sequence SEQ ID NO 1:

(SEQ ID NO: 1)

ACCGAGAGAGATTCGCACTCAATATTCAAGAGATATTGAGTGCGAATCTC
TCTCTTTTTATCGATG;
Human, murine and rat genome targeting sequence
SEQ ID NO: 2:

(SEQ ID NO: 2)

ACCGAGATCGCCACCTACAGGAAATTCAAGAGATTTCCTGTAGGTGGCGA
TCTCTTTTTATCGATG.
The siRNA GAGAGAGATTCGCACTCAATA is herein
identified as SEQ ID NO: 3.
The siRNA TATTGAGTGCGAATCTCTCTC is herein
identified as SEQ ID NO: 4.
The siRNA GAGATCGCCACCTACAGGAAA is herein
identified as SEQ ID NO: 5.
The siRNA TTTCCTGTAGGTGGCGATCTC is herein
identified as SEQ ID NO: 6.
Preferred shRNA designed to silence the murine
gene encoding vimentin are:

(SEQ ID NO: 7)

ACCGAATGGTACAAGTCCAGGTTTGTTCAAGAGACAAACTTGGACTTGTA
CCATTCTTTTTCTCGAGG.

(SEQ ID NO: 8)

ACCGAGAGAAATTGCAGGAGGAGATTCAAGAGATCTCCTCCTGCAATTTC
TCTCTTTTTCTCGAGG
The siRNA GAATGGTACAAGTCCAGGTTTG is herein
identified as SEQ ID NO: 9.
The siRNA CAAACTTGGACTTGTACCATTC is herein
identified as SEQ ID NO: 10.
The siRNA GAGAGAAATTGCAGGAGGAGA is herein
identified as SEQ ID NO: 11.
The siRNA TCTCCTCCTGCAATTTCTCTC is herein
identified as SEQ ID NO: 12.

Preferred siRNA targeting the human gene encoding vimentin, described by Harborth et al. (2001), have been identified by inventors as usable, in the context of the present invention, to prevent, treat or alleviate the above mentioned disorders associated with the formation of a glial scar. The sense and antisense sequences of these siRNA molecules are indicated below:

1)

SEQ ID NO 13

| | |
|---|---|
| sense sequence: | GAAUGGUACAAAUCCAAGU(dTdT) |

SEQ ID NO 14

| | |
|---|---|
| antisense sequence: | ACUUGGAUUUGUACCAUU(dTdT) |
| 2) | |

SEQ ID NO 15

| | |
|---|---|
| sense sequence: | AUGGAAGAGAACUUUGCCG(dTdT) |

SEQ ID NO 16

| | |
|---|---|
| antisense sequence: | CGGCAAAGUUCUCUUCCAU(dTdT) |
| 3) | |

SEQ ID NO 17

| | |
|---|---|
| sense sequence: | UACCAAGACCUGCUCAAUG(dTdT) |

SEQ ID NO 18

| | |
|---|---|
| antisense sequence: | CAUUGAGCAGGUCUUGGUA(dTdT) |

### Lentivirus Vectors

Inventors demonstrate that the above described nucleic acid molecule, capable of mediating RNA interference, can be safely, efficiently and durably expressed in target cells by using appropriate expression vectors herein described.

An appropriate expression vector is a non replicative lentivirus comprising a lentiviral genome comprising a nucleic acid sequence producing at least one functional micro RNA (miRNA), at least one functional short-hairpin RNA (shRNA) and/or at least one functional short interfering RNA (siRNA), said siRNA being preferably derived from said shRNA, said miRNA, shRNA and siRNA being designed to silence the expression of a gene that encodes a protein of the astrocyte cytoskeleton, said lentivirus being pseudotyped for the selective transfer of the lentiviral genome into cells of the central nervous system, preferably into glial cells, even more preferably into astrocytes.

In a particular embodiment, the non replicative lentivirus of the invention, comprises a lentiviral genome as previously described further comprising a second nucleic acid sequence producing at least one functional miRNA, at least one functional shRNA and/or at least one functional siRNA, preferably derived from said shRNA, said miRNA, shRNA and siRNA being designed to silence the expression of a gene encoding a different protein of the astrocyte cytoskeleton.

Lentiviruses are complex retroviruses capable of transducing cells which are not mitotically active, such as cells of the nervous system, in particular certain cell subpopulations of the central nervous system. These viruses include in particular Human Immunodeficiency Virus type 1 (HIV-1), Human Immunodeficiency Virus type 2 (HIV-2), Simian Immunodeficiency Virus (SIV), Feline Immunodeficiency Virus (FIV), Equine Infectious Anaemia Virus (EIAV), Bovine Immunodeficiency Virus (BIV), Visna Virus of sheep (VISNA) and Caprine Arthritis-Encephalitis Virus (CAEV). A preferred lentivirus according to the present invention is selected in the above mentioned list of viruses.

Like other retroviruses, lentiviruses have gag, pol and env genes flanked by two LTR (Long Terminal Repeat) sequences. Each of these genes encodes many proteins which are initially expressed in the form of a single precursor polypeptide. The gag gene encodes the internal structural proteins (capsids and nucleocapsids). The pol gene encodes the reverse transcriptase, the integrase and the protease. The env gene encodes the viral envelope glycoprotein and also contains a cis-acting RRE (Rev Responsive Element) responsible for exporting the viral RNA out of the nucleus. The 5′ and 3′ LTR sequences serve to promote the transcription and the polyadenylation of the viral RNAs. The LTR contains all the other cis-acting sequences necessary for viral replication. Sequences necessary for the reverse transcription of the genome (tRNA primer binding site) and for encapsidation of the viral RNA into particles (site Ψ) are adjacent to the 5′ LTR. If the sequences necessary for encapsidation (or for packaging of the retroviral RNA into infectious virions) are absent from the viral genome, the genomic RNA will not be actively encapsidated.

The construction of lentiviral vectors for gene transfer applications has been described, for example, in patents U.S. Pat. No. 5,665,577, EP 386 882, U.S. Pat. No. 5,981,276 and U.S. Pat. No. 6,013,516 or else in patent application WO 99/58701.

The vectors used in the present invention are non replicative, in other words they comprise a defective lentiviral genome, i.e., a genome in which at least one of the gag, pol and env genes has been inactivated or deleted. These vector genomes are encapsidated in a protein particle composed of the structural lentiviral proteins and in particular of the envelope glycoprotein.

The recombinant lentiviruses according to the invention are thus genetically modified in such a way that certain genes constituting the native infectious virus are eliminated and replaced with a nucleic acid sequence of interest to be introduced into the target cells. After adsorption of the virus on the cell membrane, said virus injects its nucleic acid into the cell and, after reverse transcription, said nucleic acid can integrate into the genome of the host cell. The genetic material thus transferred is then transcribed and possibly translated into proteins inside the host cell. When the lentiviral vector is a non integrative lentiviral vector, the genetic material transferred in host cells is present in episomal forms, named 1 LTR or 2 LTR circles.

A preferred non replicative lentivirus herein described is a lentivirus deprived of any lentiviral coding sequence. It is also deleted of the enhancer region of the U3 region of the LTR3′. Particularly preferred lentiviral vector are pseudotyped vectors that allow targeting of a cell population of the central nervous system. The term “pseudotyping” denotes a recombinant virus comprising an envelope different from the wild-type envelope. In the context of the present invention, the vectors express an envelop protein which direct the vector to various cells, including the cells of the Central Nervous System.

An appropriate envelope glycoprotein is a vesiculovirus envelope glycoprotein such as the envelope glycoprotein of the vesicular stomatitis virus (VSV). This envelope exhibits advantageous characteristics, such as resistance to ultracentrifugation and a very broad tropism. Unlike other envelopes, such as those of the conventional retroviruses (amphotropic and ecotropic MLV retroviruses or HIV gp120, but also many others), the VSV glycoprotein is not labile after ultracentrifugation. This makes it possible to concentrate the viral supernatants and to obtain high infectious titres. Moreover, this envelope confers on the virions a very broad tropism, in particular in vitro, allowing the infection of a very large number of cell types, including cells of the central nervous system, in particular glial cells such as astrocytes. The receptor for this envelope is thought to be a phosphatidylserine motif present at the surface of many cells of various species. VSV-G is an example of such a VSV envelop glycoprotein.

Preferred vectors allow targeting of glial cells, preferably glial cells of the astrocyte type. These pseudotyped viral vectors are useful for the transfer and the expression in vitro, ex vivo and in vivo of nucleic acid sequences of interest preferentially within astrocytes.

The term “preferentially” should be understood to mean that the lentiviruses according to the invention target essentially astrocytes but are, nevertheless, capable of transfecting other cell types, such as other glial cells of the central nervous system. Other nerve cell subpopulations which may be targeted by vectors of the invention are, for example, microglial cells, endothelial cells or oligodendrocytes.

Preferred envelopes allowing the preferential targeting of glial cells of the astrocyte type are lyssavirus envelopes, in particular a virus envelop of the rabies virus serogroup selected from the group consisting of Rabies (RAB); Duvenhague (DUV), European Bat type 1 (EB-1), European Bat type 2 (EB-2), Kotonkan (KOT), Lagos Bat (LB), Mokola (MOK), Obodhiang (OBD) and Rochambeau (RBU), or any chimeric composition of these envelopes. In a preferred embodiment, inventors use lentiviral vectors, for example of the HIV type, pseudotyped with an envelope of the PV (rabies virus) or MOK (Mokola virus) type. Other envelope glycoproteins that can be used to allow preferential targeting of glial cells are alphaviruses envelopes, in particular the Ross River virus (RRV) glycoprotein, and arenaviridae envelopes, in particular the Lymphocytic choriomeningitis virus (LCMV) glycoprotein.

In a particular embodiment, the lentivirus comprises a lentiviral genome comprising, between wild type or modified (att mutants) LTR5′ and LTR3′ sequences, a Psi (Ψ) encapsidation sequence, at least one coding sequence producing at least one functional miRNA, at least one functional shRNA, and/or at least one functional siRNA preferably derived from said shRNA, and optionally: a promoter, a sequence enhancing RNA nuclear import such as the cppT-CTS, a sequence enhancing RNA nuclear export, a transcription regulation element, and/or a mutated integrase.

The above mentioned promoter can be a viral or a cellular promoter.

A preferred cellular promoter usable, in the context of the present invention, to express a shRNA, may be selected from the group consisting of U6, H1 and 7SK RNA polymerase III promoter.

A preferred viral promoter usable, in the context of the present invention, to express a miRNA targeting GFAP or vimentine, may be selected from the group consisting of CMV, TK, RSV LTR polymerase II promoter.

A preferred cellular promoter usable, in the context of the present invention, to express a miRNA targeting GFAP or vimentine, may be selected from the group consisting of PGK, Rho, EF1α, GFAP, Vimentin, Nestin, S100β.

In a particular embodiment, the promoter is a transactivator induced promoter as further explained below, preferably comprising a plurality of transactivator binding sequences operatively linked to the nucleic acid sequence producing shRNA.

A particularly preferred sequence enhancing the RNA nuclear import is the lentiviral cPPT CTS (flap) sequence from HIV-1. Other sequences, usable in the context of the present invention, enhancing RNA nuclear import are lentiviral cPPT CTS sequences from (HIV-2, SIV, FIV, EIAV, BIV, VISNA and CAEV). A particularly preferred sequence enhancing the RNA nuclear export is a sequence comprising the HIV-1 REV response element (RRE) sequence. Another sequence, usable in the context of the present invention, which enhances the RNA nuclear export, is the CTE sequence (Oh et al 2007). Preferred posttranscriptional regulation elements may be selected from Woodchuck hepatitis virus responsive element (WPRE), APP UTR5′ region and TAU UTR3′. A preferred regulation element will be an insulator sequence selected from the group consisting of, for example, MAR, SAR, S/MAR, scs and scs'.

A preferred lentivirus is non integrative (EP 1761635). Such a lentivirus comprises a mutated integrase in order to limit the risk of insertion mutagenesis. Preferably the integrase comprises a mutation in at least one of its basic region (N, L and/or Q regions, preferably L and Q regions) and/or catalytic region. The lentivirus integration can also be silenced by mutating the att sequence of the LTRs, by mutating the CA motif of the att sequence (Nightinghale et al. 2006).

The lentiviral vectors according to the invention can be prepared in various ways, notably by transient transfection(s) into producer cells (or using stable producer cell lines) and/or by means of helper viruses.

The method according to the invention comprises, according to a particularly preferred embodiment, the transfection of a combination of a minimum of three plasmids in order to produce a recombinant virion or a recombinant retrovirus.

A first plasmid provides the lentiviral vector genome comprising the cis-acting viral sequences necessary for the correct functioning of the viral cycle. Such sequences include preferably one or more lentiviral LTRs, a Psi (ψ) packaging sequence, reverse transcription signals, a promoter and/or an enhancer and/or polyadenylation sequences. In this vector, the LTRs can also be modified so as to improve the expression of the transgene or the safety of the vector. Thus, it is possible to modify, for example, the sequence of the 3′ LTR by eliminating the U3 region [modified sequence herein identified as LTR(ΔU3)] (see WO 99/31251). One can also introduce the transgene cassette (promoter+transgene) in the vectors genome between the LTRs, or in place of the U3 region of the LTR 3′.

According to a particular embodiment of the invention, it is a vector plasmid comprising a recombinant lentiviral genome of sequence LTR-psi-Promoter-transgene-LTR which allows expression of the vector RNA which will be encapsidated in the virions.

A preferred vector plasmid comprises a recombinant lentiviral genome of sequence LTR-psi-flap-Promoter-transgene-LTR wherein flap designates the sequence cPPT CTS enhancing the ARN nuclear import.

Another preferred vector plasmid comprises a recombinant lentiviral genome of sequence LTR-psi-flap-Promoter-transgene-WPRE-LTR, wherein flap designates the sequence cPPT CTS which improves the transduction of non dividing cells, and in particular which enhances the ARN nuclear import, and wherein WPRE (Woodchuck hepatitis virus responsive element) is a transcription regulation element, advantageously used to enhance the transgene expression level.

In the present invention, the transgene or nucleic acid of interest produces at least one functional nucleic acid molecule capable of mediating RNA interference, preferably at least one functional miRNA, at least one functional short-hairpin RNA (shRNA), or at least one functional siRNA derived from said shRNA, said nucleic acid molecule being designed to silence the expression of at least one target gene, in particular a gene that encodes a protein of the astrocyte cytoskeleton, said protein being selected preferably, from GFAP and vimentin.

The transgene is typically placed under the control of a transcriptional promoter. A promoter that is particularly useful in the context of the present invention has a transcription machinery that is compatible with mammalian genes, can be compatible with expression of genes from a wide variety of species, preferably has a high basal transcription rate, recognizes termination sites with a high level of accuracy. A preferred promoter will preferably be sufficient to direct the transcription of a distally located sequence, which is a sequence linked to the 3′ end of the promoter sequence in a cell.

Since long poly A tails compromise the silencing effect of shRNAs, their expression is appropriately driven by RNA polymerase III which recognizes a run of 5T residues as a stop signal and does not therefore require a poly A sequence to terminate transcription.

Suitable promoters include, for example, RNA polymerase (pol) III promoters including, but not limited to, the (human and murine) U6 promoters, the (human and murine) H1 promoters, and the (human and murine) 7SK promoters. In addition, a hybrid promoter also can be prepared that contains elements derived from, for example, distinct types of RNA polymerase (pol) III promoters. Modified promoters that contain sequence elements derived from two or more naturally occurring promoter sequences can be combined by the skilled person to effect transcription under a desired set of conditions or in a specific context. For example, the human and murine U6 RNA polymerase (pol) III and H1 RNA pol III promoters are well characterized and useful for practicing the invention. One skilled in the art will be able to select and/or modify the promoter that is most effective for the desired application and cell type so as to optimize modulation of the expression of one or more genes. The promoter sequence can be one that does not occur in nature, so long as it functions in a eukaryotic cell, preferably a mammalian cell.

Expression of the transgene or nucleic acid of interest, here the at least one functional miRNA, shRNA or siRNA derived from said shRNA, may be externally controlled by treating the cell with a modulating factor, such as doxycycline, tetracycline or analogues thereof. Analogues of tetracycline are for example chlortetracycline, oxytetracycline, demethylchloro-tetracycline, methacycline, doxycycline and minocycline. Conditional suppression of genes may indeed be important for therapeutic applications by allowing time and/or dosage control of the treatment or by permitting to terminate treatments at the onset of unwanted side effects.

Reversible gene silencing may be implemented using a transactivator induced promoter together with said transactivator. Such a transactivator induced promoter comprises control elements for the enhancement or repression of transcription of the transgene or nucleic acid of interest producing miRNA, shRNA and/or siRNA. Control elements include, without limitation, operators, enhancers and promoters. A transactivator inducible promoter, in the context of the present invention, is transcriptionally active when bound to a transactivator, which in turn is activated under a specific set of conditions, for example, in the presence or in the absence of a particular combination of chemical signals, preferably by a modulating factor selected for example from the previous list.

The transactivator induced promoter may be any promoter herein mentioned which has been modified to incorporate transactivator binding sequences, such as several tet-operon sequences, for example 7 tet-operon sequences, preferably in tandem. Such sequences can for example replace the functional recognition sites for Staf and Oct-1 in the distal sequence element (DSE) of the U6 promoter, preferably the human U6 promoter.

Advantageously, the transactivator induced promoter comprises a plurality of transactivator binding sequences operatively linked to the nucleic acid sequence producing shRNAs.

The transactivator may be provided by a nucleic acid sequence, in the same expression vector or in a different expression vector, comprising a modulating factor-dependent promoter operatively linked to a sequence encoding the transactivator. The term “different expression vector” is intended to include any vehicle for delivery of a nucleic acid, for example, a virus, plasmid, cosmid or transposon. Suitable promoters for use in said nucleic acid sequence include, for example, constitutive, regulated, tissue-specific or ubiquitous promoters, which may be of cellular, viral or synthetic origin, such as CMV, RSV, PGK, EF1α, NSE, synapsin, β-actin, GFAP.

A preferred transactivator according to the present invention is the rtTA-Oct.2 transactivator composed of the DNA binding domain of rtTA2-M2 and of the Oct-2^{Q}(Q→A) activation domain.

Another preferred transactivator according to the present invention is the rtTA-Oct.3 transactivator composed of the DNA binding domain of the Tet-repressor protein (E. coli) and of the Oct-2^{Q}(Q→A) activation domain.

Both are described in patent application WO 2007/004062.

As used herein, the term “operatively linked” means that the elements are connected in a manner such that each element can serve its intended function and the elements, together can serve their intended function. In reference to elements that regulate gene expression, “operatively linked” means that a first regulatory element or coding sequence in a nucleotide sequence is located and oriented in relation to a second regulatory element or coding sequence in the same nucleic acid so that the first regulatory element or coding sequence operates in its intended manner in relation with the second regulatory element or coding sequence.

When the lentivirus comprises a transactivator induced promoter, said lentivirus may further advantageously comprise a WPRE which is able to enhance the expression of the transactivator.

A second plasmid, for trans-complementation, provides a nucleic acid encoding the protein products of the gag and pol lentiviral genes. These proteins are derived from a lentivirus and preferably originate from HIV, in particular HIV-1. The second plasmid is devoid of encapsidation sequence, of sequence encoding an envelope and, advantageously, is also devoid of lentiviral LTRs. As a result, the sequences encoding gag and pol proteins are advantageously placed under control of a heterologous promoter, for example a viral, cellular, etc. promoter, which may be constitutive or regulated, weak or strong. It is preferably a trans-complementing plasmid comprising a sequence CMV-Δpsi-gag-pol-Δenv-PolyA. This plasmid allows the expression of all the proteins necessary for the formation of empty virions, except the envelope glycoproteins. It is understood that the gag and pol genes may also be carried by different plasmids.

A third plasmid provides a nucleic acid which allows the production of the chosen envelope (env) glycoprotein. This envelope may be chosen from the envelopes mentioned above, in particular an envelope of a rhabdovirus, more particularly of a lyssavirus, even more preferably an envelop of the Mokola virus. This vector is preferentially devoid of all lentiviral sequences, encapsidation sequence and of sequences encoding gag or pol and, advantageously, is also devoid of lentiviral LTRs.

Advantageously, the three vectors used do not contain any homologous sequence sufficient to allow a recombination. The nucleic acids encoding gag, pol and env may advantageously be cDNAs prepared according to conventional techniques, from sequences of the viral genes available in the prior art and on databases.

For the production of the non replicative lentiviruses, the vectors described above are introduced into competent cells and the viruses produced are harvested. The cells used may be any competent cell, preferably mammalian cell, for example animal or human cell, which is non pathogenic. Mention may, for example, be made of 293 cells, embryonic cells, fibroblasts, muscle cells, etc.

A preferred method for preparing a non replicative recombinant lentivirus, according to the invention, comprises transfecting a population of competent cells with a combination of vectors as described above, and recovering the viruses thus produced.

A particularly advantageous method for producing lentiviruses capable of silencing in vivo the expression of a gene that encodes a protein of the astrocyte cytoskeleton, in particular in human astrocytes, comprises transfection of competent cells with:

a) a vector plasmid comprising a sequence, as described previously, such as LTR-psi-Promoter-transgene-LTR(ΔU3),
b) a trans-complementing plasmid comprising a sequence CMV-Δpsi-gag-pol-Δenv-PolyA, and
c) an envelope plasmid comprising a sequence CMV-env-PolyA, the envelope being preferably an envelope of the rabies virus serogroup.

The lentiviruses of the invention may also be prepared, as explained previously, from an encapsidation cell line producing one or more gag, pol and env proteins.

### Therapeutic Uses

The lentiviruses according to the invention may be used for preparing a composition intended for gene transfer into astrocytes in vivo or ex vivo.

The lentiviruses according to the invention may in particular be used for preparing a pharmaceutical composition intended to prevent, treat or alleviate a nervous system, in particular a central nervous system (CNS), disorder in an animal subject, preferably a mammal, in particular a human.

Another subject of the invention lies in the combined use of several identical or different lentiviruses as herein described, for the purpose of transferring and expressing several identical or different miRNAs, shRNAs and/or siRNA in the cells of the nervous system, in particular in glial cells, preferably in astrocytes. The combined use may comprise sequential administrations of the various viruses, or a simultaneous administration.

The lentiviruses of the invention may further allow the transport and the expression, within nerve cells, of at least one nucleic acid encoding for example a compound selected from a growth factor such as FGF, a trophic factor such as GDNF, BDNF, NGF, NT-3, a cytokine, a colony stimulating factor, an anticancer agent, a toxin, an enzyme, a neurotransmitter or a precursor thereof, a component of the extracellular matrix (ECM) such as N-CAM, PAS-NCAM, laminin, fibronectin, N-cadherin, a growth associated protein such as GAP-43, CAP-23 etc., enhancing the activity of the at least one functional nucleic acid molecule capable of mediating RNA interference also produced, and/or enhancing the prophylactic or therapeutic effect thereof.

Herein provided is a method for modulating, preferably repressing expression of a target gene. Such a method may be used for preventing, treating or alleviating a nervous system disorder, in particular a central nervous system (CNS) disorder, in an animal subject, in particular a mammal, preferably a human, comprising administering to said animal (i) a pharmaceutical composition comprising a non replicative lentivirus comprising a lentiviral genome comprising a nucleic acid sequence producing at least one functional nucleic acid molecule capable of mediating RNA interference, preferably at least one functional miRNA, at least one functional shRNA, or at least one functional siRNA derived from said shRNA, said shRNA being designed to silence the expression of a gene that encodes a protein of the astrocyte cytoskeleton, said lentivirus being preferably pseudotyped for the selective transfer of the lentiviral genome into nervous cells, in particular cells of the central nervous system, preferably glial cells, even more preferably astrocytes, and (ii) a pharmaceutically acceptable carrier or excipient.

In a particular embodiment, invention also relates to a method as described previously, wherein said method comprises two steps consisting in successively contacting a cell with a lentivirus according to the present invention or a composition comprising such a lentivirus, and with a modulating factor such as tetracycline, as previously described, and wherein said two steps may be inverted.

The target gene expression repression can be reversed upon withdrawal of the modulating factor or upon interruption of the modulating factor treatment or on the contrary upon administration, adjunction or application of a modulating factor, depending, as explained previously, on the transactivator used. Such a method can be realized in a dose- and time-dependent manner.

The nervous system disorder is preferably a central nervous system disorder. Such a SNC disorder may be a brain or spinal cord trauma or a stroke.

The disorder may also be any condition associated with the formation of a glial scar, for example a brain or spinal cord trauma or stroke, or a neurodegenerative disease, including, but not limited to Parkinson's disease, Huntington's disease, Alzheimer's disease, Amyotrophic Lateral Sclerosis (ALS), Spinal Muscular Atrophy (SMA), multiple sclerosis, temporal lobe epilepsy, lupus erythematosus and human immunodeficiency virus dementia.

The doses of vector may be adjusted by the skilled person depending on the route of administration, tissue, vector, compound, etc.

The composition is advantageously administered at a rate of about 0.01 to 10⁴ ng of P24 capsidic protein preferably between about 5 to 400 ng of capsidic protein P24. The lentiviruses may be purified and conditioned in any suitable composition, solution or buffer, comprising pharmaceutically acceptable an excipient, vehicle or carrier, such as a saline, isotonic, buffered solution such as Mannitol 20%, optionally combined with stabilizing agents such as isogenic albumin or any other stabilizing protein, glycerol, etc., and also adjuvants such as polybrene or DEAE dextrans, etc.

Various protocols may be used for the administration, such as simultaneous or sequential administration, single or repeated administration, etc., which may be adjusted by the skilled person.

A lentiviral vector can be used that provide for transient expression of siRNA molecules in the case of non integrative lentiviral vectors. Such vectors can be repeatedly administered as necessary.

The pharmaceutical composition containing the lentivirus according to the invention may be administered to a patient intracerebrally, intraspinally or systemically given the particular tropism of the pseudotyped lentiviral vectors, in particular for nervous cells such as glial cells of the astrocyte type.

Thus, it may be an administration given intracerebrally, intraspinally, i.e., directly in the medullar parenchyma, intra-striatally, intra-venously, intra-arterially. Preferred modes of injection are intracerebral injection, intraspinal injection, intrathécale injection.

Also herein provided is a kit for expressing a nucleic acid as herein described, designed to silence the expression of a gene encoding a protein of the astrocyte cytoskeleton, comprising (i) at least one non replicative lentivirus according to the present invention comprising a lentiviral genome comprising a nucleic acid sequence producing said nucleic acid designed to silence the expression of a gene, said lentivirus being pseudotyped for the selective transfer of the lentiviral genome into cells of the central nervous system, and optionally (ii) a leaflet providing guidelines.

Also provided is a cloning kit comprising:

a) a vector plasmid comprising a sequence, as described previously, such as LTR-psi-Promoter-transgene-LTR(ΔU3),
b) a trans-complementing plasmid comprising a sequence CMV-Δpsi-gag-pol-Δenv-PolyA,
c) an envelope plasmid comprising a sequence CMV-env-PolyA, the envelope being preferably an envelope of the rabies virus serogroup, and optionally
d) a leaflet providing guidelines.

Further aspects and advantages of this invention are disclosed in the following experimental section, which should be regarded as illustrative and not limiting the scope of this application in any way.

### Experimental Part

1. Development of Lentiviral Vectors that Allow Inhibition of GFAP and Vimentin Expression.

### a) Description of the Lv-shGFAP and Lv-shVIM Vectors:

In order to drive a powerful and long-term inhibition of GFAP and Vimentin expression in reactive astrocytes, inventors developed lentiviral vectors that express shRNAs directed against GFAP and Vimentin.

Inventors screened the cDNA encoding murine GFAP and vimentine to determinate oligonucleotides sequences that are capable of efficiently suppressing the expression of both proteins. Using a plasmid vector, candidate sequences were expressed as short hairpin RNAs (shRNAs) in HEK 293 T cells cotransfected with a plasmid expressing the fusion protein GFAP-GFP (or Vimentin-GFP). Efficient shRNAs caused the destruction of the unique GFAP-GFP mRNA (or Vimentin-GFP) and yielded thereby a decline in GFP fluorescence as was followed by Fluorescence Activated Cell Sorting (FACS). Results of the screening are represented on FIG. 3.

Two shRNAs were obtained that decreased the expression of GFAP-GFP by 90%. The shGFAP sequence SEQ ID NO: 1 matches with mouse genome, and the shGFAP sequence SEQ ID NO: 2 matches with mouse, rat and human genome. Moreover two shRNAs were obtained that decreased the expression of Vimentin-GFP by 60% (SEQ ID NO 3 and 4 match with murine genome). These sequences match with mouse genome. Human siVIM sequence has been described by Harborth et al. (2001).

To express these shRNAs in primary cultured astrocytes and in vivo, inventors then constructed lentiviral vectors that deliver shRNA against GFAP or vimentine (these vectors were respectively called Lv-shGFAP and Lv-shVIM). These vectors are derived from the lentivirus HIV-1. Selected shRNA sequence was inserted downstream from the human U6 promoter into precursor plasmid <<pFlap>>. The vectors derived from this precursor plasmid contain the central HIV-1 Flap sequence, which facilitate transduction of non-dividing cells (Zennou et al., 2001). Furthermore, the Woodchuck Hepatitis Virus Responsive Element (WPRE, Zufferey et al., 1999) was included into this precursor plasmid to protect the RNA genome of the vector from RNAi mediated degradation during the production of the vector particles. Using this WPRE element, several teams have produced high-titred stocks of lentiviral vectors expressing shRNAs (Rubinson et al. 2003, Tiscornia et al. 2003). For safety reasons the U3 promoter region is deleted from the 3′ LTR so that the vector is non replicative (Zufferey et al., 1998). In order to control transduction efficiency in vitro and in vivo, inventors also inserted the coding sequence of the fluorescent protein E-GFP, downstream from the ubiquitous PGK promoter.

Lentivirus vector particles were produced by transient co-transfection of HEK 293-T cells by the precursor plasmid, an encapsidation plasmid (p8.9) and an envelope expression plasmid. Lentiviruses produced by this method could be pseudotyped by different kind of envelopes. Lv-shGFAP and Lv-shVIM were pseudotyped with VSV envelope, which allow ubiquitous cell transduction, or with Mokola envelope, which target glial cells in vivo (Mammeri et al., in preparation).

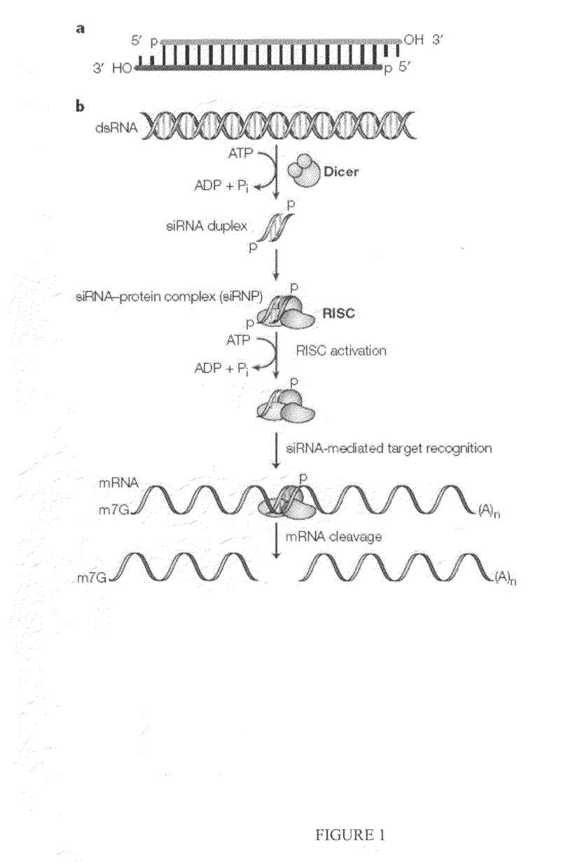

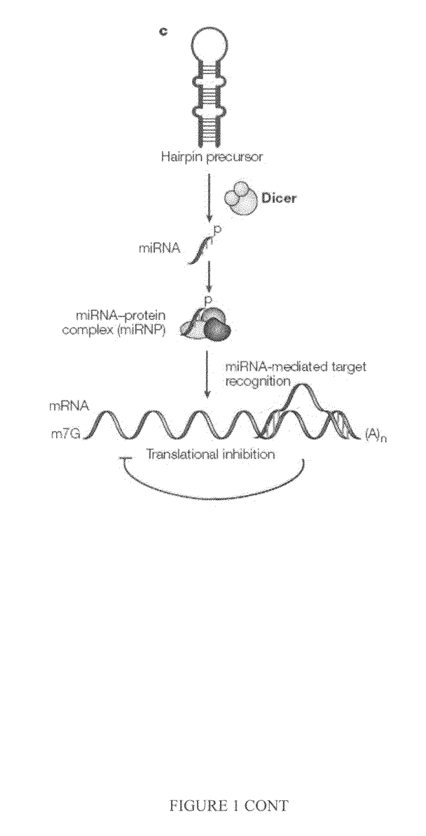

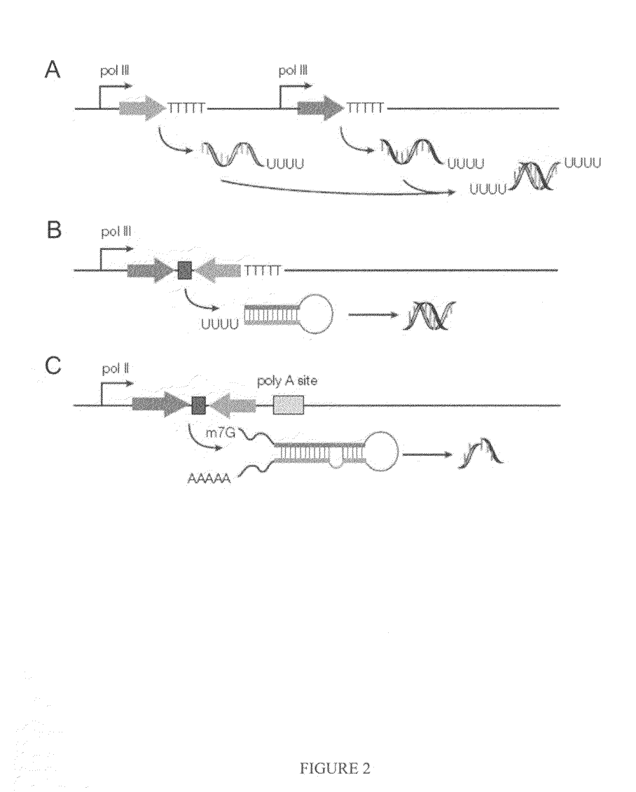

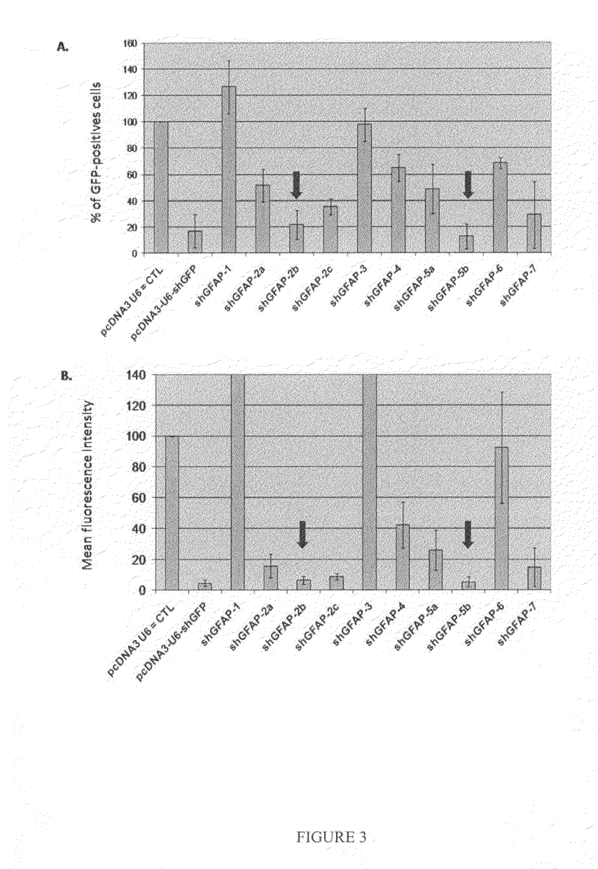

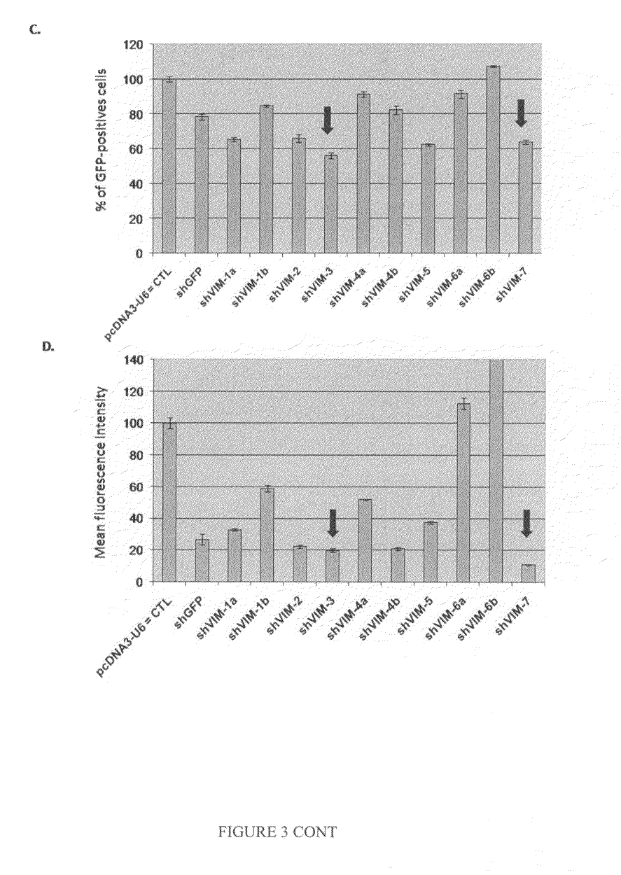

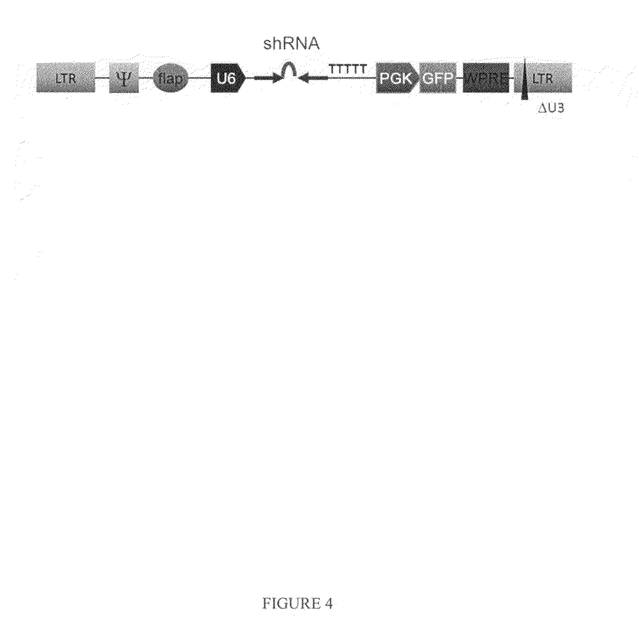

The structure of these vectors are described in FIG. 4.

### b) In Vitro Characterization of the Lentiviral Vectors:

Before their use in vivo, the lentiviral vectors Lv-shGFAP and Lv-shVIM were first tested in three different in vitro models, in order to evaluate their ability to reduce GFAP and vimentine expression, to modulate astrocytic response to a lesion, and to promote neuronal survival and neurite growth.

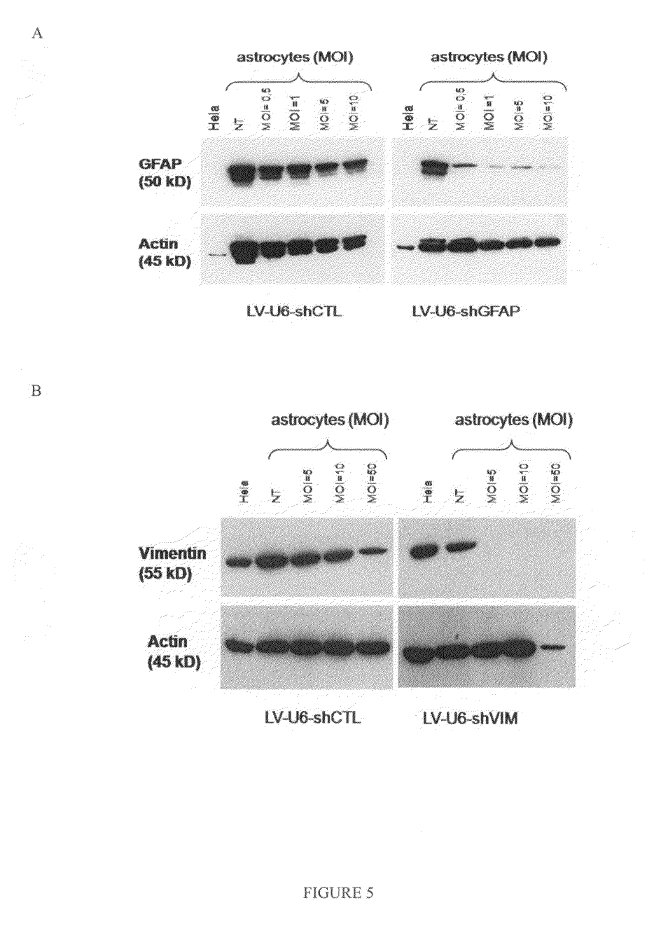

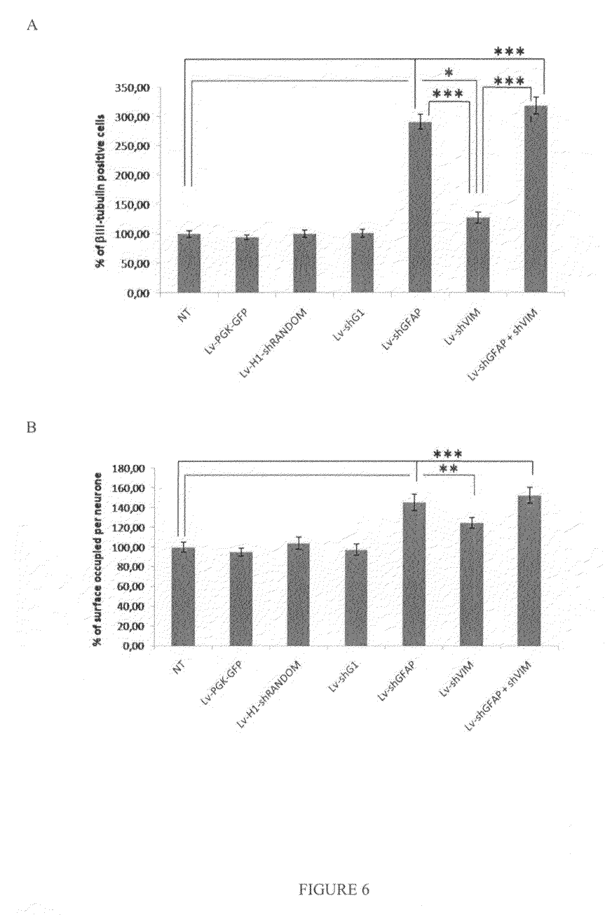

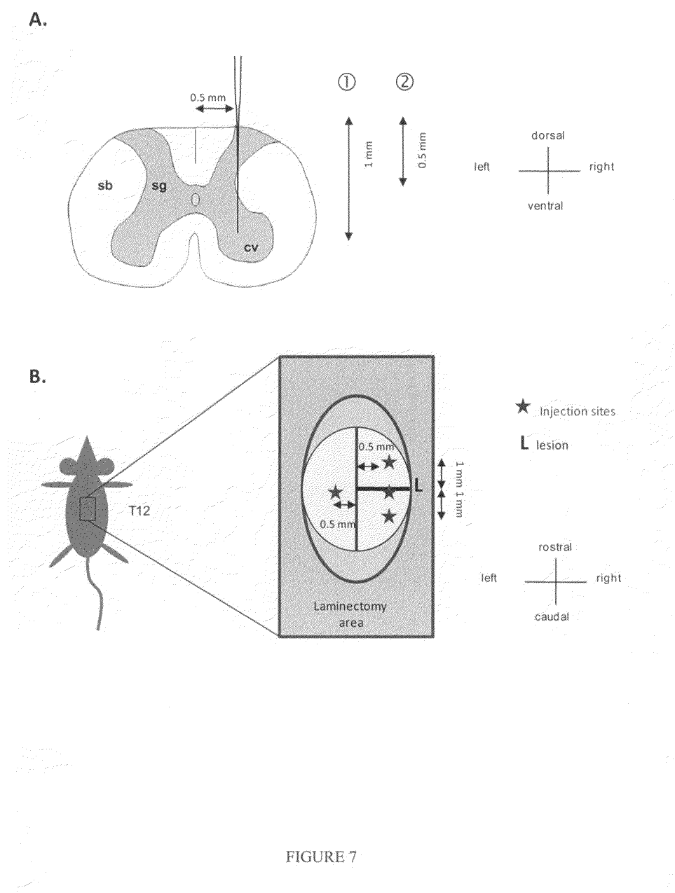

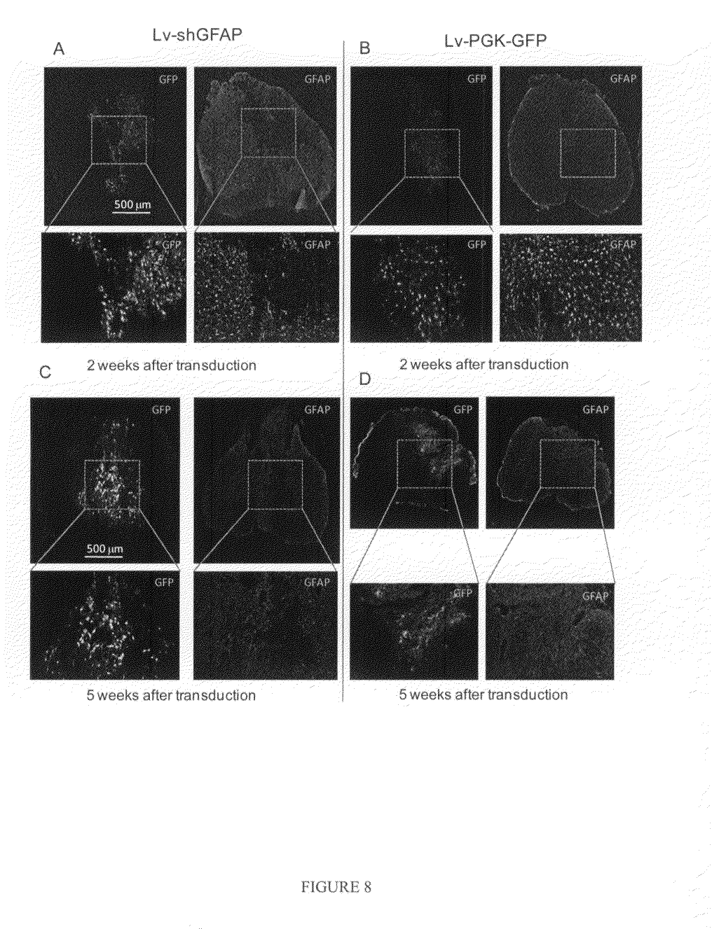

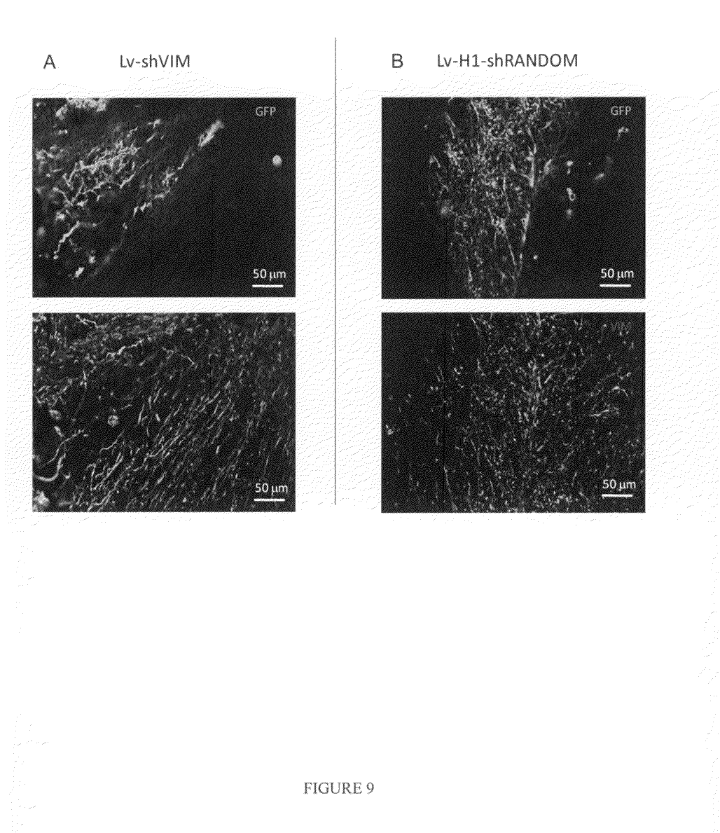

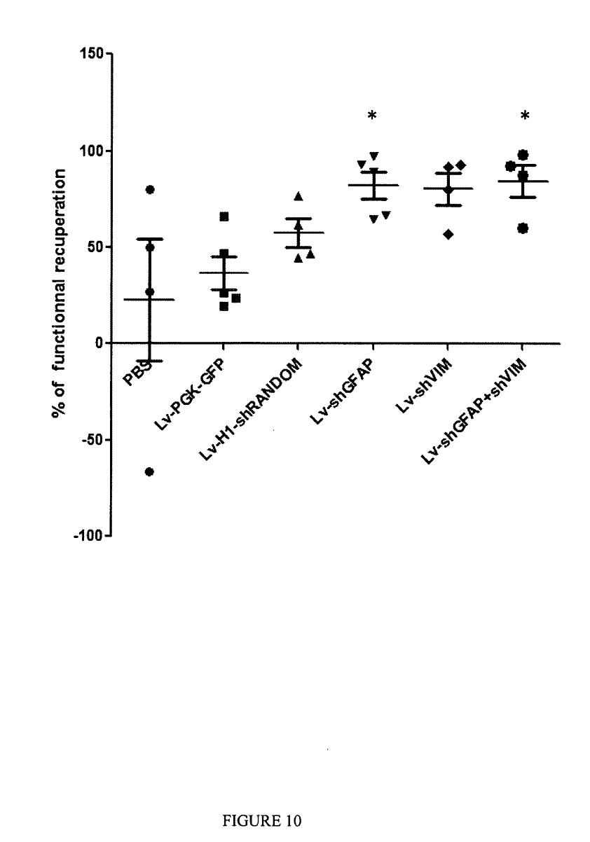

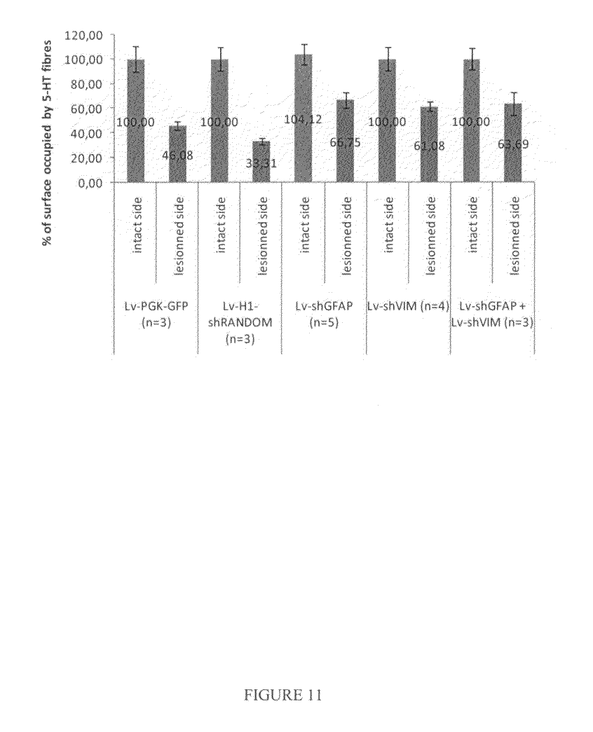

In a first model of primary cultured astrocytes derived from the brains of newborn mice, the ability of the lentiviral vectors Lv-shGFAP and Lv-shVIM to mediate silencing of endogenous GFAP and Vimentin was evaluated. These cultures are good models of reactive astrocytes (Bignami and Dahl, 1989; Privat et al., 1995). The cells were transduced with different quantities of the vectors, and the expression of GFAP and vimentine has been monitorised by Western Blot analysis. Two weeks after transduction, an up to 90% reduction of the expression of the GFAP and vimentine was obtained in comparison with controls, as revealed in FIG. 5.

The lentiviral vectors Lv-shGFAP and Lv-shVIM were then applied into an astrocytes-neurons coculture model, in order to specify whether these vectors affects neuronal survival and/or neurite outgrowth. In this model, cortical neurons were prepared from 14 days old mice embryos and were cultured on neonatal astrocytes previously transduced with Lv-shGFAP, Lv-shVIM and different control vectors (which are represented by the following vectors: Lv-PGK-GFP, Lv-shRANDOM and Lv-shG1). After one week of coculture, the cells were fixed and βIII-tubulin immunostaining was performed in order to detect the neuronal pericaryons and neurites. Inventors demonstrate that the lentiviral vector Lv-shGFAP, alone or associated with the vector Lv-shVIM, induce a significant increase in neuronal survival and in neurite outgrowth (see FIG. 6). These results establish the ability of the Lv-shGFAP lentiviral vector to promote neuronal survival/axonal regrowth in an in vitro model of glial reactivity.

In a third model of in vitro scratch wound, inventors evaluated the response of reactive astrocytes, transduced with the Lv-shGFAP and Lv-shVIM vectors. In this model astrocytic monolayers, previously transduced with the different lentiviral vectors, were scratched with a sterile pipette tip. After 2 days, one week and 2 weeks of incubation, cells were fixed and GFP immunostaining was performed in order to visualize the transduced cells. In comparison to controls, cells transduced with Lv-shGFAP and Lv-shVIM fail to repair the scratch wound at the different times of fixation. These results show that Lv-shGFAP and Lv-shVIM lentiviral induce a modulation of the astrogliosis phenotype and a reduction in the astrocytic scarring process in vitro.

### 2. Application of the Lentiviral Vectors Allowing RNAi-Mediated Inhibition of GFAP And Vimentin in CNS Pathologies.

### a) Spinal Cord Injury

In order to promote axonal regeneration and functional recovery after acute traumatic injury, inventors developed a therapeutic strategy based on the injections of Lv-shGFAP and Lv-shVIM lentiviral vectors into an in vivo model of spinal cord injury. This model consists in complete unilateral hemisection of the spinal cord in adult C57BL/6 mice. In these animals, they injected directly Lv-shGFAP and Lv-shVIM lentiviral vectors in the medullar parenchyma. They first developed an injection procedure in order to transduce a maximal number of reactive astrocytes around the lesion area, in both rostro-caudal and dorso-ventral axis. More specifically they determined injection parameters that allow transduction of precise spinal cord areas which are covered by neuronal tracts implicated in locomotion control, such as the corticospinal tract or the serotonergic fibers in the ventral horn. These injection parameters are (i) 4 injection sites as described in FIG. 7, (ii) 2 injections sub-sites that refer to two different injection depth: 1 mm and 0.5 mm (iii) a volume of 1 μl per site, (iv) an injection speed of 0.2 μl per minute (v) a dose of 100 ng P24 of lentiviral vector per site, (vi) intraperitoneal administration of mannitol 20% 15 minutes before vectors injection.

Technically, the hemisection is performed at thoracic level T12, in order to promote axonal regrowth upstream from the Central Pattern Generator (CPG), which is located at lumbar level L2-L3 in the mouse. The injection procedure comprises 4 injections sites around the lesion. In each injection site, the lentiviral vectors are applied along 2 sub-sites, which correspond to two different depth levels of injection (respectively 0.5 and 1 mm). Lentiviral vectors are injected dorsally, in the vertical axis, by using tapered glass capillaries, with a velocity of 0.2 microliters per minute. The total volume of injected lentiviral vectors was 1 μl per site, and the total amount of vector injected per site was 500 000 viral particles (˜100 ng P24). Moreover Mannitol 20% was administrated in the animal by intraperitoneal injection 15 minutes before the lentiviral vectors injection in order to increase the transduction efficiency, as it was previously described for adenoviral vectors (Ghodsi et al., 1999).

This injection procedure allows the transduction of a large area around the lesion site which can be extended on 500 μm in the dorso-ventral axis. Inventors were able to block the GFAP and Vimentin expression in a large region around the lesion, which lead to a significantly reduced glial reactivity.

Inventors then confirmed in vivo the inhibition of the endogenous surexpression of GFAP and Vimentin by the Lv-shGFAP and Lv-shVIM vectors after injury. As presented in FIG. 8, GFAP expression is decreased in the spinal cord area transduced with the lv-shGFAP lentiviral vector. As presented in FIG. 9, Vimentin is decreased in the spinal cord with the lv-shVIM lentiviral vector. These results show clearly the ability of the vectors Lv-shGFAP and Lv-shVIM to reduce the glial scar in vivo In further studies different series of hemisectioned mice were treated with different lentiviral vectors including the Lv-shGFAP and Lv-shVIM vectors, and with control vectors. In these animals inventors analyse the formation of the glial scar, the axonal regeneration around the lesion site and finally the functional recovery, using the same methods and behavioural tests used to characterize the double mutant (GFAP −/−, vim −/−) mice (Menet et al., 2003).

The functional recovery of the treated animals was evaluated by the behavioural test named grid walk. As illustrated in FIG. 10, in this test, the animals treated with the Lv-shGFAP, the Lv-shVIM, or both vectors, present the best scores of recuperation, when compared to controls. The recuperation score is significantly increased for animals treated with the Lv-shGFAP vector or with both Lv-shGFAP and Lv-shVIM vectors when datas are analyzed with the statistical Mann-Whitney test (*p<0.05) Additionally, an automated analysis of locomotion was performed before and one month after the surgery, on the same animals. Moreover in the same animals on which the behavioural test was performed, inventors also monitored immunohistochemical analyses in order to detect axonal regeneration of serotonergic fibres in the ventral horn, that are implicated in locomotion. The results show that ventral horns of animals treated with the Lv-shGFAP and Lv-shVIM contain more serotonergic fibers than control animals. (see FIG. 11)

These results of in vivo application show that the Lv-shGFAP vector, alone or associated with the Lv-shVIM vector, allow sustained reduction of GFAP expression in vivo and promote functional recovery after spinal cord lesion

### b) Parkinson Disease

Inventors applied the Vimentin and GFAP KO approach to a degenerative pathology, namely Parkinson disease.

For that purpose, control mice as well as mice knocked out (KO) for GFAP, Vimentin or both genes were injected with the toxin 6-hydroxy-dopamine (6-OH-DA) in the striatum, in order to induce a partial degeneration of dopaminergic neurons of the substantia nigra. Mice were then blindly followed for one month with a battery of functional tests and then sacrificed to analyze the anatomical substrate and in particular the possible regeneration of dopaminergic axons.

In a study performed on a group of six animals comprising GFAP KO mice, double GFAP/Vimentin KO mice or control mice, inventors evaluated the survival of dopaminergic neurons after 6-OH-DA lesion of the substantia nigra.

GFAP KO and double GFAP/vimentine KO mice present no decrease of the dopaminergic neurons number or a significant increase reaching up to 60% of the dopaminergic neurons number, in the injured side compared to the non-injured side. On the contrary control mice present a significant decrease of 40 to 60% of the dopaminergic neuron number, in the injured side compared to the non-injured side. These results show an increased plasticity in the GFAP KO mice and in the double GFAP/vimentin KO mice which is related to a permissive glial substrate. The demonstration that Lv-shGFAP and Lv-shVIM can modulate glial permissivity in vitro and in vivo, seriously suggests that these vectors can reduce the dopaminergic neuron loss in animal models of Parkinson disease.

### REFERENCES

Bartsch, U., Bandtlow, C. E., Schnell, L., Bartsch, S., Spillmann, A. A., Rubin, B. P., Hillenbrand, R., Montag, D., Schwab, M. E., and Schachner, M. (1995). Lack of evidence that myelin-associated glycoprotein is a major inhibitor of axonal regeneration in the CNS. Neuron 15, 1375-1381.
Bradbury, E. J., Moon, L. D., Popat, R. J., King, V. R., Bennett, G. S., Patel, P. N., Fawcett, J. W., and McMahon, S. B. (2002). Chondroitinase ABC promotes functional recovery after spinal cord injury. Nature 416, 636-640.
Bregman, B. S., Kunkel-Bagden, E., Schnell, L., Dai, H. N., Gao, D., and Schwab, M. E. (1995). Recovery from spinal cord injury mediated by antibodies to neurite growth inhibitors. Nature 378, 498-501.
Brosamle, C., Huber, A. B., Fiedler, M., Skerra, A., and Schwab, M. E. (2000). Regeneration of lesioned corticospinal tract fibers in the adult rat induced by a recombinant, humanized IN-1 antibody fragment. J Neurosci 20, 8061-8068.
Brummelkamp, T. R., Bernards, R., and Agami, R. (2002). A system for stable expression of short interfering RNAs in mammalian cells. Science 296, 550-553.
Caroni, P., and Schwab, M. E. (1988). Antibody against myelin-associated inhibitor of neurite growth neutralizes nonpermissive substrate properties of CNS white matter. Neuron 1, 85-96.
Dykxhoorn D. M., Novina C. D., Sharp Philip A. (2003) Killing the messenger: short RNAs that silence gene expression. Nat Rev Mol Cell Biol. 4, 457-467.
El Maarouf, A., Petridis, A. K., and Rutishauser, U. (2006). Use of polysialic acid in repair of the central nervous system. Proceedings of the National Academy of Sciences of the United States of America 103, 16989-16994.
Fawcett, J. W. (2006). Overcoming inhibition in the damaged spinal cord. Journal of neurotrauma 23, 371-383.
Fawcett, J. W., and Asher, R. A. (1999). The glial scar and central nervous system repair. Brain research bulletin 49, 377-391.
Freund, P., Schmidlin, E., Wannier, T., Bloch, J., Mir, A., Schwab, M. E., and Rouiller, E. M. (2006). Nogo-A-specific antibody treatment enhances sprouting and functional recovery after cervical lesion in adult primates. Nature medicine 12, 790-792.
Freund, P., Wannier, T., Schmidlin, E., Bloch, J., Mir, A., Schwab, M. E., and Rouiller, E. M. (2007). Anti-Nogo-A antibody treatment enhances sprouting of corticospinal axons rostral to a unilateral cervical spinal cord lesion in adult macaque monkey. The Journal of comparative neurology 502, 644-659.
Ghodsi, A., Stein, C., Derksen, T., Martins, I., Anderson, R. D., and Davidson, B. L. (1999). Systemic hyperosmolality improves beta-glucuronidase distribution and pathology in murine MPS VII brain following intraventricular gene transfer. Experimental neurology 160, 109-116.
Kim, J. E., L1, S., GrandPre, T., Qiu, D., and Strittmatter, S. M. (2003). Axon regeneration in young adult mice lacking Nogo-A/B. Neuron 38, 187-199.
Harborth, J., Elbashir, S. M., Bechert, K., Tuschl, T., and Weber, K. (2001). Identification of essential genes in cultured mammalian cells using small interfering RNAs. J Cell Sci 114, 4557-4565.
Liebscher, T., Schnell, L., Schnell, D., Scholl, J., Schneider, R., Gullo, M., Fouad, K., Mir, A., Rausch, M., Kindler, D., et al. (2005). Nogo-A antibody improves regeneration and locomotion of spinal cord-injured rats. Annals of neurology 58, 706-719.
Menet, V., Gimenez y Ribotta, M., Chauvet, N., Drian, M. J., Lannoy, J., Colucci-Guyon, E., and Privat, A. (2001). Inactivation of the glial fibrillary acidic protein gene, but not that of vimentin, improves neuronal survival and neurite growth by modifying adhesion molecule expression. J Neurosci 21, 6147-6158.
Menet, V., Prieto, M., Privat, A., and Gimenez y Ribotta, M. (2003). Axonal plasticity and functional recovery after spinal cord injury in mice deficient in both glial fibrillary acidic protein and vimentin genes. Proceedings of the National Academy of Sciences of the United States of America 100, 8999-9004.
Nightingale S J, Hollis R P, Pepper K A, Petersen D, Yu X J, Yang C, Bahner I, Kohn D B. Transient gene expression by nonintegrating lentiviral vectors. Mol Ther. 2006 June; 13(6):1121-32.
Oh T., Bajwa A., Jia G., Park F. Lentiviral vector design using alternative RNA export elements. (2007) Retrovirology 5; 4:38
Pekovic, S., Filipovic, R., Subasic, S., Lavrnja, I., Stojkov, D., Nedeljkovic, N., Rakic, L., and Stojiljkovic, M. (2005). Downregulation of glial scarring after brain injury: the effect of purine nucleoside analogue ribavirin. Annals of the New York Academy of Sciences 1048, 296-310.
Schnell, L., and Schwab, M. E. (1990). Axonal regeneration in the rat spinal cord produced by an antibody against myelin-associated neurite growth inhibitors. Nature 343, 269-272.
Silver, J., and Miller, J. H. (2004). Regeneration beyond the glial scar. Nature reviews 5, 146-156.
Simonen, M., Pedersen, V., Weinmann, 0., Schnell, L., Buss, A., Ledermann, B., Christ, F., Sansig, G., van der Putten, H., and Schwab, M. E. (2003). Systemic deletion of the myelin-associated outgrowth inhibitor Nogo-A improves regenerative and plastic responses after spinal cord injury. Neuron 38, 201-211.
Sjobeck, M., and Englund, E. (2003). Glial levels determine severity of white matter disease in Alzheimer's disease: a neuropathological study of glial changes. Neuropathol Appl Neurobiol 29, 159-169.
Teismann, P., and Schulz, J. B. (2004). Cellular pathology of Parkinson's disease: astrocytes, microglia and inflammation. Cell Tissue Res 318, 149-161.
Yiu, G., and He, Z. (2006). Glial inhibition of CNS axon regeneration. Nature reviews 7, 617-627.
Zheng, B., Ho, C., L1, S., Keirstead, H., Steward, 0., and Tessier-Lavigne, M. (2003). Lack of enhanced spinal regeneration in Nogo-deficient mice. Neuron 38, 213-224.

1. A non replicative lentivirus comprising a lentiviral genome comprising a nucleic acid sequence producing at least one functional micro RNA (miRNA), at least one functional short-hairpin RNA (shRNA) and/or at least one functional short interfering RNA (siRNA), said miRNA, shRNA and siRNA being designed to silence the expression of a gene that encodes a protein of the astrocyte cytoskeleton, said lentivirus being pseudotyped for the selective transfer of the lentiviral genome into cells of the central nervous system.

2. The lentivirus according to claim 1, wherein the lentiviral genome further comprises a second nucleic acid sequence producing at least one functional miRNA, at least one functional shRNA and/or at least one functional siRNA, said miRNA, shRNA and siRNA being designed to silence the expression of a gene that encodes a different protein of the astrocyte cytoskeleton.

3. The lentivirus according to claim 1, wherein the protein of the astrocyte cytoskeleton is selected from GFAP and vimentin.

4. The lentivirus according to claim 3, wherein, when the shRNA is designed to silence a gene encoding GFAP, said shRNA is:
(SEQ ID NO: 1)
(i)
ACCGAGAGAGATTCGCACTCAATATTCAAGAGATATTGAGTGCGAATC
TCTCTCTTTTTATCGATG,
or
(SEQ ID NO: 2)
(ii)
ACCGAGATCGCCACCTACAGGAAATTCAAGAGATTTCCTGTAGGTGGC
GATCTCTTTTTATCGATG,
and, wherein, when the shRNA is designed to silence a gene encoding vimentin, said shRNA is:
(SEQ ID NO: 7)
(i)
ACCGAATGGTACAAGTCCAGGTTTGTTCAAGAGACAAACTTGGACTTG
TACCATTCTTTTTCTCGAGG,
or
(SEQ ID NO: 8)
(ii)
ACCGAGAGAAATTGCAGGAGGAGATTCAAGAGATCTCCTCCTGCAATT
TCTCTCTTTTTCTCGAGG.

5. The lentivirus according to claim 3, wherein, when the siRNA is designed to silence a gene encoding GFAP, said siRNA is:

| | | |
|---|---|---|
| (i) | GAGAGAGATTCGCACTCAATA, | (SEQ ID NO: 3) |
| (ii) | TATTGAGTGCGAATCTCTCTC, | (SEQ ID NO: 4) |
| (iii) | GAGATCGCCACCTACAGGAAA | (SEQ ID NO: 5) |
| or | | |
| (iv) | TTTCCTGTAGGTGGCGATCTC, | (SEQ ID NO: 6) |

and, wherein, when the siRNA is designed to silence a gene encoding vimentin, said siRNA is:

| | | |
|---|---|---|
| (i) | GAATGGTACAAGTCCAGGTTTG, | (SEQ ID NO: 9) |
| (ii) | CAAACTTGGACTTGTACCATTC, | (SEQ ID NO: 10) |
| (iii) | GAGAGAAATTGCAGGAGGAGA | (SEQ ID NO: 11) |
| or | | |
| (iv) | TCTCCTCCTGCAATTTCTCTC. | (SEQ ID NO: 12) |

6. The lentivirus according to claim 1, wherein the lentivirus is selected from the group consisting of Human Immunodeficiency Virus type 1 (HIV-1), Human Immunodeficiency Virus type 2 (HIV-2), Simian Immunodeficiency Virus (SIV), Feline Immunodeficiency Virus (FIV), Equine Infectious Anaemia Virus (EIAV), Bovine Immunodeficiency Virus (BIV), Visna Virus of sheep (VISNA) and Caprine Arthritis-Encephalitis Virus (CAEV).

7. The lentivirus according to claim 1, wherein the lentivirus is deprived of any lentiviral coding sequence and of the enhancer region of the U3 region of the LTR3′.

8. The lentivirus according to claim 1, wherein said lentivirus is pseudotyped with a lyssavirus envelope, in particular with a virus envelop of the rabies virus serogroup selected from the group consisting of Rabies (RAB); Duvenhague (DUV), European Bat type 1 (EB-1), European Bat type 2 (EB-2), Kotonkan (KOT), Lagos Bat (LB), Mokola (MOK), Obodhiang (OBD) and Rochambeau (RBU), or any chimeric composition of these envelopes.

9. The lentivirus according to claim 1, wherein said lentivirus is pseudotyped with an alphavirus envelope, in particular with a virus envelop of the Ross River Virus (RRV).

10. The lentivirus according to claim 1, wherein said lentivirus is pseudotyped with an areanviridae envelope, in particular with a virus envelop of the Lymphocytic choriomeningitis virus (LCMV).

11. The lentivirus according to claim 1, wherein said lentivirus is pseudotyped with a vesiculovirus envelope.

12. The lentivirus according to claim 1, wherein the lentiviral genome comprises, between LTR3′ and LTR5′ sequences, a lentiviral ψ encapsidation sequence, a coding sequence producing shRNA, and optionally a promoter, a sequence enhancing RNA nuclear import, a sequence enhancing RNA nuclear export, a transcription regulation element, and/or a mutated integrase.

13. The lentivirus according to claim 12, wherein the promoter is a viral promoter or a cellular promoter.

14. The lentivirus according to claim 13, wherein the promoter is a cellular promoter allowing expression of a shRNA, selected from the group consisting of U6, H1 and 7SK RNA polymerase III promoter.

15. The lentivirus according to claim 13, wherein the promoter is a viral promoter allowing expression of a miRNA, selected from the group consisting of CMV, TK, RSV LTR polymerase II promoter.

16. The lentivirus according to claim 13, wherein the promoter is a cellular promoter allowing expression of a miRNA, selected from the group consisting of PGK, Rho, EF1α, GFAP, Vimentin, Nestin, S100β polymerase II promoter.

17. The lentivirus according to claim 12, wherein the promoter is a transactivator induced promoter that modulates RNA interference, said transactivator induced promoter comprising a plurality of transactivator binding sequences operatively linked to the nucleic acid sequence producing shRNA.

18. The lentivirus according to claim 17, wherein the transactivator induced promoter is a tetracycline-dependent transactivator selected from the rtTA-Oct.2 transactivator composed of the DNA binding domain of rtTA2-M2 and of the Oct-2^{Q}(Q→A) activation domain, and the rtTA-Oct.3 transactivator composed of the DNA binding domain of the E. coli Tet-repressor protein and of the Oct-2^{Q}(Q→A) activation domain.

19. The lentivirus according to claim 12, wherein the sequence enhancing the ARN nuclear import is lentiviral cPPT CTS (Flap) sequence.

20. The lentivirus according to claim 12, wherein the sequence enhancing the ARN nuclear export comprises HIV-1 REV response element (RRE) sequence.

21. The lentivirus according to claim 12, wherein the sequence enhancing the nuclear export comprises the CTE element

22. The lentivirus according to claim 12, wherein the transcription regulation element is selected from Woodchuck hepatitis virus responsive element (WPRE), APP UTR5′ region, TAU UTR3′, and insulators MAR, SAR, S/MAR, scs and scs' sequence.

23. The lentivirus according to claim 12, wherein the integrase comprises a mutation in at least one of its basic region and/or catalytic region responsible for the lentivirus to be non integrative.

24. A method for preventing or treating a central nervous system (CNS) disorder in an animal, comprising administering to said animal a pharmaceutical composition comprising a non replicative lentivirus comprising a lentiviral genome comprising a nucleic acid sequence producing at least one functional miRNA, at least one functional shRNA and/or at least one functional siRNA, said miRNA, shRNA and siRNA being designed to silence the expression of a gene that encodes a protein of the astrocyte cytoskeleton, said lentivirus being pseudotyped for the selective transfer of the lentiviral genome into cells of the central nervous system, and a pharmaceutically acceptable excipient.

25. The method according to claim 24, wherein said animal is a human.

26. The method according to claim 24, wherein the disorder is a brain or spinal cord trauma or a stroke.

27. The method according to claim 24, wherein the disorder is a neurodegenerative disease selected from Parkinson's disease, Huntington's chorea, Alzheimer's disease, Amyotrophic Lateral Sclerosis (ALS) and Spinal Muscular Atrophy (SMA).

28. The method according to claim 24, wherein the administration of the pharmaceutical composition is by intracerebral, intraspinal or intrathecal injection.

29. A kit for expressing a nucleic acid designed to silence the expression of a gene encoding a protein of the astrocyte cytoskeleton, comprising at least one non replicative lentivirus comprising a lentiviral genome comprising a nucleic acid sequence producing at least one functional miRNA, at least one functional shRNA and/or at least one functional siRNA, said miRNA, shRNA and siRNA being designed to silence the expression of a gene that encodes a protein of the astrocyte cytoskeleton, said lentivirus being pseudotyped for the selective transfer of the lentiviral genome into cells of the central nervous system.
